(19) United States
(12) Patent Application Publication
(10) Pub. No.: US 2018/0344296 A1
(43) Pub. Date: Dec. 6, 2018

(54) METHOD AND APPARATUS FOR ENDOMETRIAL SCREENING

(71) Applicant: BORIS PETRIKOVSKY, GREAT NECK, NY (US)

(72) Inventor: BORIS PETRIKOVSKY, GREAT NECK, NY (US)

(21) Appl. No.: 15/615,314

(22) Filed: Jun. 6, 2017

(51) Int. Cl. A61B 10/00; G01N 33/574; G01N 33/50; A61F 13/84

(52) U.S. Cl. CPC A61B 10/0045; G01N 33/57442; G01N 33/5005; A61F 13/472; G01N 2800/36; A61B 2010/0074; A61F 13/84

(57) ABSTRACT

Disclosed is a menses cell capture device having at least one endometrial cell filter having a filter substrate defining a plurality of pores sized and arrayed so as to be effective in capturing endometrial cells, and a collection platform, such as a sanitary napkin or tampon, supporting said endometrial cell filters at least partially in the path of a menstrual flow in a configuration effective in collecting endometrial cells discharged in the menstrual flow.

Also disclosed is a method of screening for endometrial disorders in a subject by providing an endometrial cell filter at least partially positioned in a menstrual flow of said subject, recovering and identifying endometrial cells collected in the endometrial cell filter, and determining whether any of said endometrial cells present with an endometrial disorder, such as inter alia atypical hyperplasia and adenocarcinoma.

### CROSS-REFERENCE TO RELATED APPLICATION

This application claims the benefit of U.S. Provisional Application No. 62/418,797, filed Nov. 8, 2016, the disclosures of which are incorporated by reference herein in their entirety.

### BACKGROUND OF THE DISCLOSURE

### Field of the Disclosure

This disclosure relates to endometrial screening by way of utilizing a menses cell capture device to capture endometrial cells during menstruation which are then analyzed for any endometrial disorders and malignancies such as, for example, infections, endometrial hyperplasia, and endometrial adenocarcinoma among others.

### BACKGROUND OF THE INVENTION

The uterus 700, has an inner lining called the endometrium 720 that defines the uterine cavity 750 (see FIG. 7), that may suffer any one of many disorders, such as infection or uterine cancer. A means of early detection of impending or actual cancer is especially desired given the potential consequences. Though uterine cancer is often thought of as a post-menopausal disease, it is known to strike younger women. Endometrial cancer is an adenocarcinoma (i.e., a cancer arising from glandular tissue, because the endometrium is a gland by virtue of its ability to secrete hormones critical to fetal development) that is among the most common gynecologic malignancies, affecting 50,560 women, and causing 9,325 deaths in 2013, the last year for which figures are available according to the Center for Disease Control & Prevention (see FIG. 9). The peak incidence of the disease is at the age of 60 years. It should be noted that the phrase “uterine cancer” in popular usage refers to endometrial cancer, but in the medical art also includes sarcoma, which is a cancer of the deep uterine muscle, the myometrium 74. In fact, endometrial cancer comprises about 80% of so-called “uterine cancers.” Cervical cancer is not included in “uterine cancer.”

The menstrual cycle has three phases, the menses, the proliferative, and the secretory. Estrogen is the dominant hormone of the proliferative phase, which earns its name by virtue of the proliferation (i.e., multiplication) of endometrial cells of the lining in order to replace the lining that was sloughed off during the preceding menses phase. Note that the menses and proliferative phases correspond to the follicular phase of the parallel ovarian cycle (see FIG. 10).

Surges of follicle stimulating hormone and luteinizing hormone cause ovulation, marking the end of the proliferative phase at about 14 days into the menstrual cycle and the beginning of the secretory phase, wherein progesterone levels rise to levels comparable to those of moderated estrogen levels. The presence of progesterone suppresses proliferation, that is to say cell division, of the endometrial cells. Instead, the cells of the endometrium grow larger, thereby thickening the endometrium (i.e., hypertrophy). Of concern is if the endometrium thickens by continued cell division during this phase, a condition known as atypical hyperplasia, which may be a sign of impending cancerous growth of the endometrial tissue, known as endometrial adenocarcinoma.

Atypical hyperplasia is characterized by distorted endometrial cells with enlarged nuclei (FIG. 8b). The most immediate feature apparent in endometrial adenocarcinomas is cytoplasmic eosinophilia—the strong tendency of the cancer cell's cytoplasm to take up the bright pink dye eosin, one of the five component dyes of Papanicolaou stain (FIG. 8c). If undiagnosed atypical hyperplasia has about a 25% chance to progress to adenocarcinoma. Women of menstruating age are not spared, as is reported in Evans-Metcalf ER et al. Profile of women 45 years of age and younger with endometrial cancer. Obstet Gynecol. 1998 March;91(3):349-54, wherein a cross-sectional study of 301 consecutive patients with endometrial cancer was performed, 40 of whom were 45 years old or younger. In the discussion, the authors indicate that in younger women cancer diagnosis may often be delayed by the assumption that symptoms are caused by dysfunctional uterine bleeding.

Endometrial adenocarcinoma is less common in women under 40, comprising less than 5% of all endometrial malignancies. It should be noted, however, that in some groups of young women—such as those presenting with polycystic ovaries, obesity, or diabetes—the risk of endometrial cancer is much higher.

In many cases, the development of endometrial hyperplasia and malignancy has an association with prolonged estrogen exposure. Farhi et al. Endometrial adenocarcinoma in women under 25 years of age. 1986 December;68(6):741-5, reported their experiences with 10 cases of endometrial cancer in women under 25 years of age. Some of these tumors (3 out of 10) were well differentiated malignancies, such as adenocarcinomas (glandular) and adenoacanthomas (glandular with epidermal differentiation). Treatment options included hysterectomies and curettage (scraping of tissue) with subsequent progestin therapy in three patients, one of whom later bore two children. If timely diagnosis is made, the low grade of these lesions and usual confinement to the endometrium contribute to a very favorable prognosis overall.

The current means of examining endometrial cells for atypical hyperplasia and adenocarcinoma is an endometrial biopsy wherein either a thin plastic pipette is inserted into the uterus to aspirate its contents, or a curette is used to scrape the fundus 730. A formal Pap smear method for endometrial cancer doesn't really exist except insofar as endometrial cells are occasionally randomly discovered among the cervical cells recovered during Pap smears.

It is found herein that a more convenient way of accessing and screening endometrial cells in women of menstruating age is to collect the cells from the vaginal flow during menstruation, which may be done by modification of sanitary napkins or tampons so as to convert them into effective cell capture devices. The endometrial cells may then be recovered from these menstrual cell capture devices and analyzed without any intrusive medical procedure at all.

### BRIEF DESCRIPTION OF THE DISCLOSURE

Disclosed is a menses cell capture device having at least one endometrial cell filter having a filter substrate defining a plurality of pores sized and arrayed so as to be effective in capturing endometrial cells, and a collection platform, such as a sanitary napkin or tampon, supporting said endometrial cell filters at least partially in the path of a menstrual flow in a configuration effective in collecting endometrial cells discharged in the menstrual flow.

Also disclosed is a method of screening for endometrial disorders in a subject by providing an endometrial cell filter at least partially positioned in a menstrual flow of said subject, recovering and identifying endometrial cells collected in the endometrial cell filter, and determining whether any of said endometrial cells present with an endometrial disorder, such as inter alia atypical hyperplasia and adenocarcinoma.

The method and apparatus disclosed is useful for detecting, inter alia, atypical endometrial hyperplasia and endometrial adenocarcinoma.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a cross-sectional view of a sanitary napkin embodiment of the disclosure.

FIG. 2 is a top plane view of the sanitary napkin embodiment of FIG. 1.

FIG. 3 is a cross-sectional view of another sanitary napkin embodiment of the disclosure.

FIG. 4 is a top plane view of the sanitary napkin embodiment of FIG. 3.

FIG. 5 is a cross-sectional view of a tampon embodiment of the disclosure.

FIG. 6 is a diagram of a method and test kit of the disclosure.

FIG. 7 is a cross-sectional diagram of the uterus.

FIGS. 8a, 8b, and 8c are color microscope slide photographs of stained aggregates of normal, atypical hyperplasic, and adenocarcinomic endometrial cells, respectively.

FIG. 9 is a bar graph of the top ten US cancer sites for the year 2013.

FIG. 10 is a graphic diagram of the Menstrual Cycle.

### DETAILED DESCRIPTION OF THE DISCLOSURE

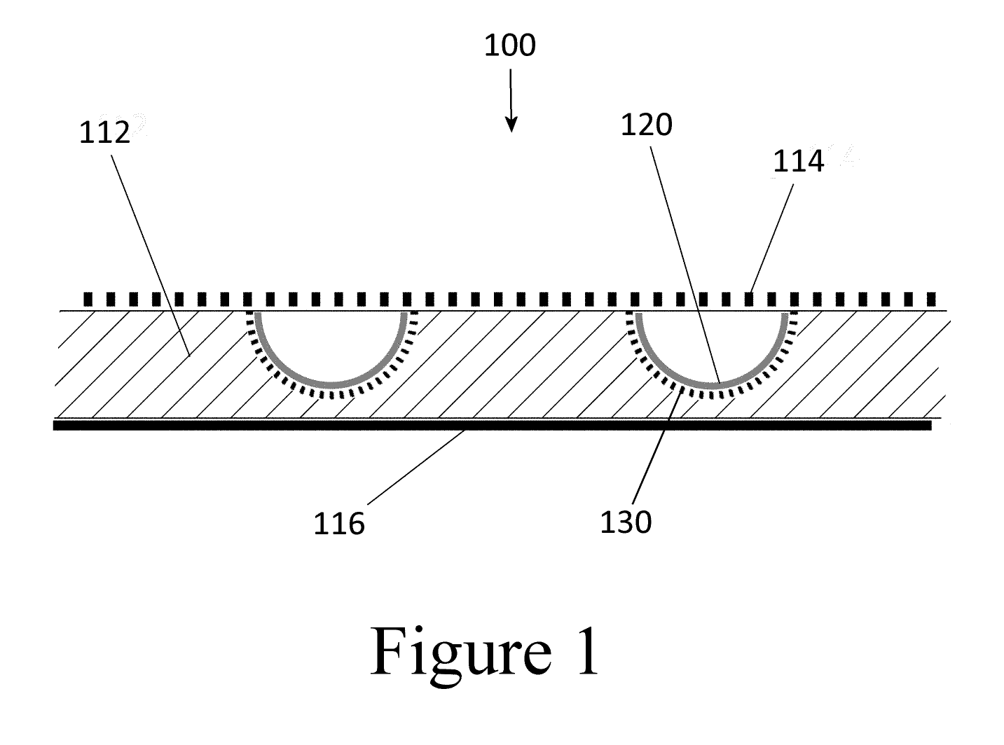

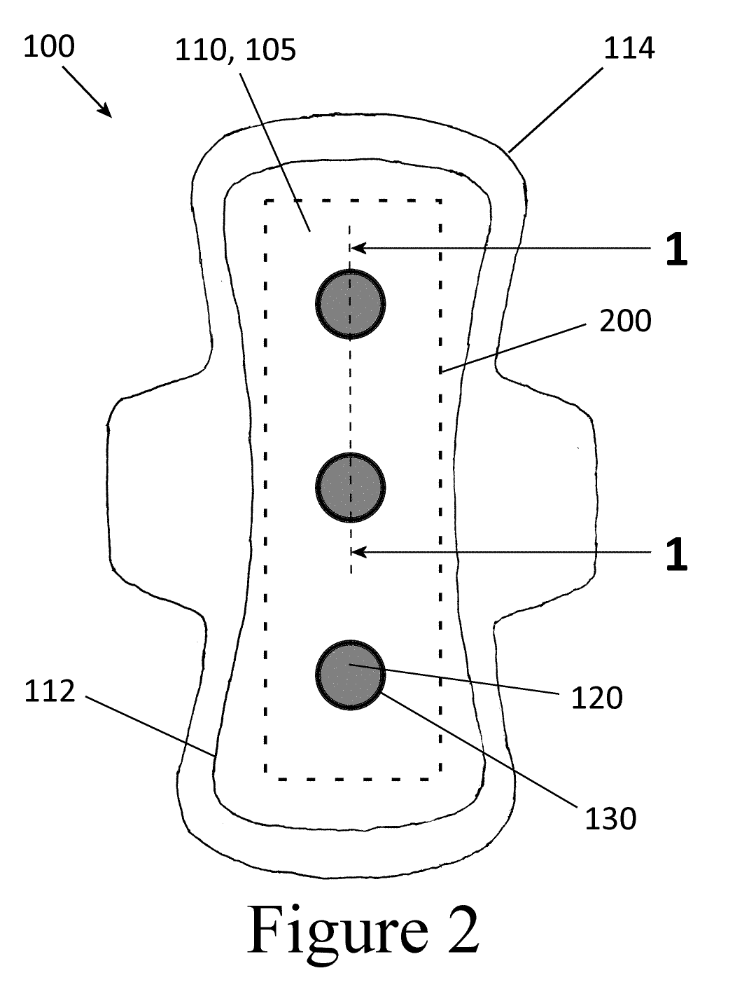

Referring to FIGS. 1 and 2, there is shown an embodiment of a menses cell capture device 100 of the disclosure, comprising a collection platform 110 and an endometrial cell filter 120. The collection platform 110 serves at least the functions of supporting and positioning the endometrial cell filter 120 at least partially in the path of menstrual flows so as to be effective in collecting endometrial cells during menses. Here, the collection platform 110 is in the nature of a sanitary napkin 105. The endometrial cell filters 120 are of a substrate material with an average pore size selected and arrayed to capture endometrial cells or cell aggregates carried by a menstrual flow from the uterus while allowing fluids and substantially smaller particulates to pass through.

As is typical with a sanitary napkin 105, there is provided a core layer 112 comprising an absorbent hydrophilic material, a porous topsheet layer 114 that is positioned against a pudendal surface of the user and facilitates the rapid transmittal of menstrual flow therethrough, and a non-porous backsheet layer 116 that may be worn in direct contact with a user's undergarment (actually, usually adhered to a panty). All three layers are usually constructed of non-woven fabrics. The average pore size of the topsheet will generally be greater than about 100 μm or 200 μm.

Often, there may also be included an optional transfer layer 200 (see FIG. 2, not shown in FIG. 1), also referred to in the art as an acquisition layer, that facilitates the rapid absorption of menstrual flow through the topsheet 114, retains the flow so as to prevent backflow through the topsheet 114 back to the user, and also distributes the menstrual flow laterally so as to spread the flow more evenly over the core layer 112. The transfer layer 200 is often directly beneath and in direct contact with the topsheet 114.

Endometrial cells are generally round or slightly cubical with a diameter of from about 10 μm to about 20 μm, averaging about 15 μm. The average filter pore size, or at least a substantial number of pores of the filters, would be in that range. That is to say that the pore size distribution of the endometrial cell filter 120 will be chosen to be effective in collecting endometrial cells carried to the filter by the menstrual flow.

In the embodiment shown, the endometrial cell filter 120 is removably held in a cell basket 130 defining pores no smaller than those of the endometrial cell filter 130. The cell basket will generally be of a rigid or semi-rigid material, such as a hydrophobic plastic polymer. Note also that while the endometrial cell filter 120 here as shown is in the shape of a lining conforming to the interior of the cell basket 130, the endometrial cell filter 120 may also be a plug of porous material stuffed in and filling the cell basket 130.

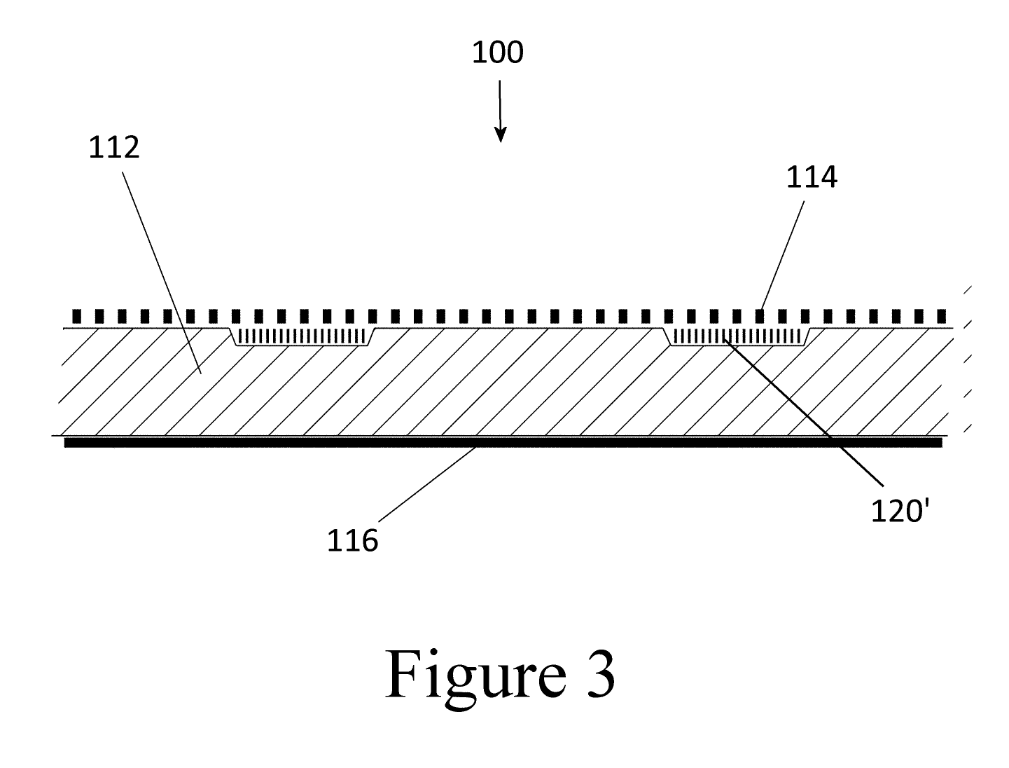

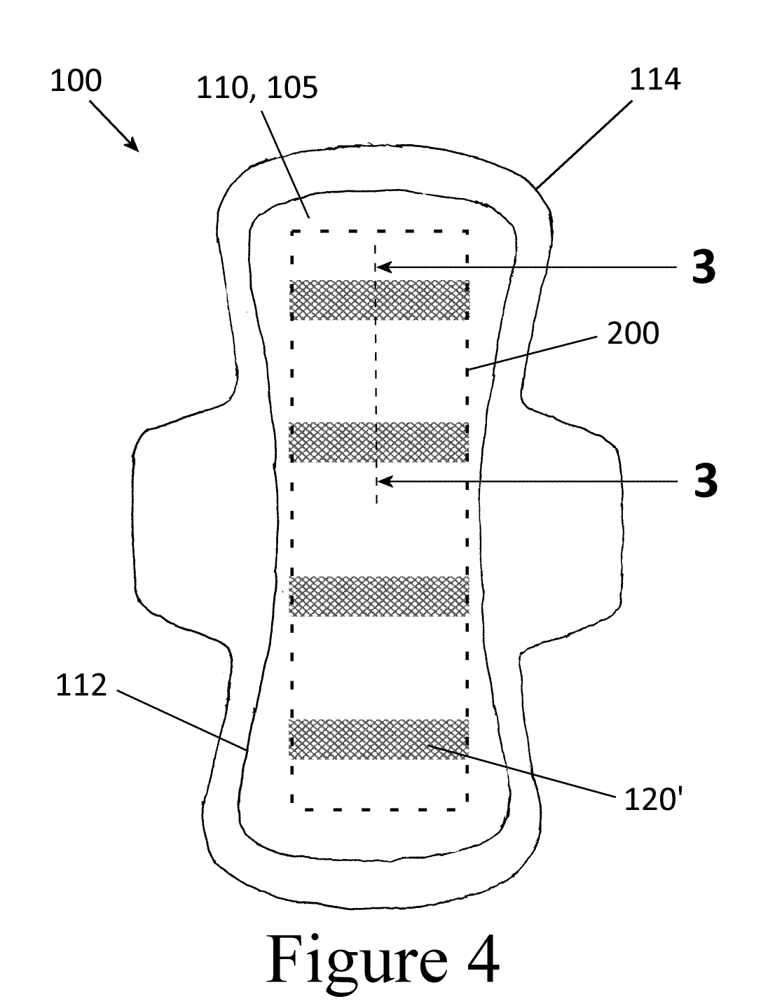

Referring now to FIGS. 3 and 4, there is shown another embodiment of a sanitary napkin 105 embodiment of a menses cell capture device 100 of the disclosure wherein one or more endometrial cell filters 120′ are provided in the form of individual elements, such as strips, removably positioned between the core layer 112 and the topsheet 114.

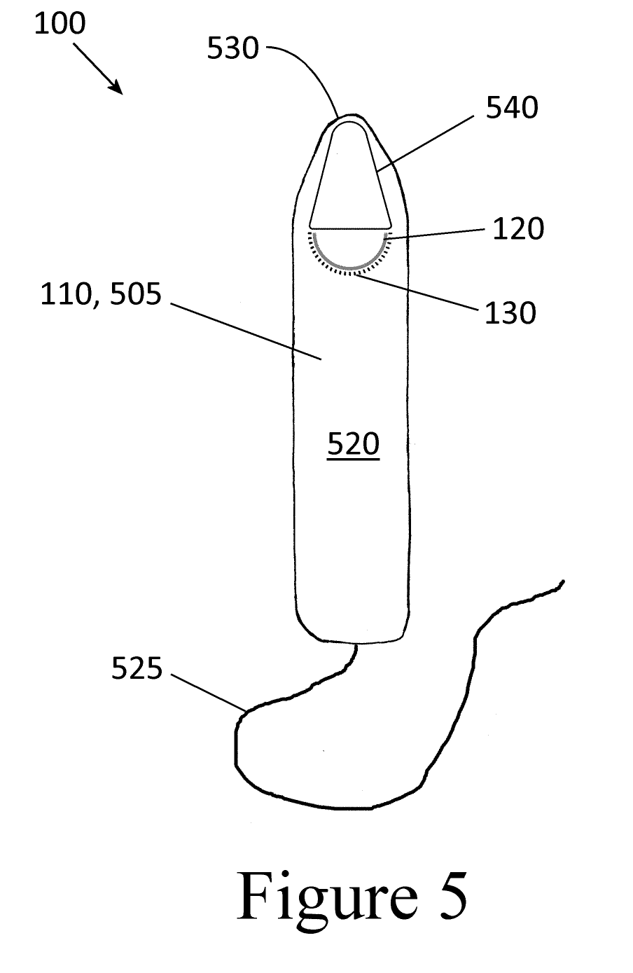

Referring to FIG. 5, there is shown a menses cell capture device 100 comprising a collection platform 110 in the nature of a tampon 505. The tampon 505 comprises a hydrophilic and absorbent shaft 520 and an insertion end 530. Downstream of the insertion end 530 is disposed the endometrial cell filter 120, here by means of the cell basket 130 configuration as shown with respect to FIGS. 1 and 2, though other means of providing an endometrial cell filter 120, 120′ may be employed. In this case, an acquisition cap 540 may be provided that has an average pore size substantially larger than about 10 μm to about 20 μm so that endometrial cells may rapidly flow to the endometrial cell filter before being diverted to the absorbent shaft 520. The acquisition cap 540 may be a rigid or semi-rigid hollow cone constructed of a perforated polymer plastic or other suitable porous material or may be a plug of absorbent material.

A string 530 may be provided for extracting the tampon 505 from a vaginal canal 790.

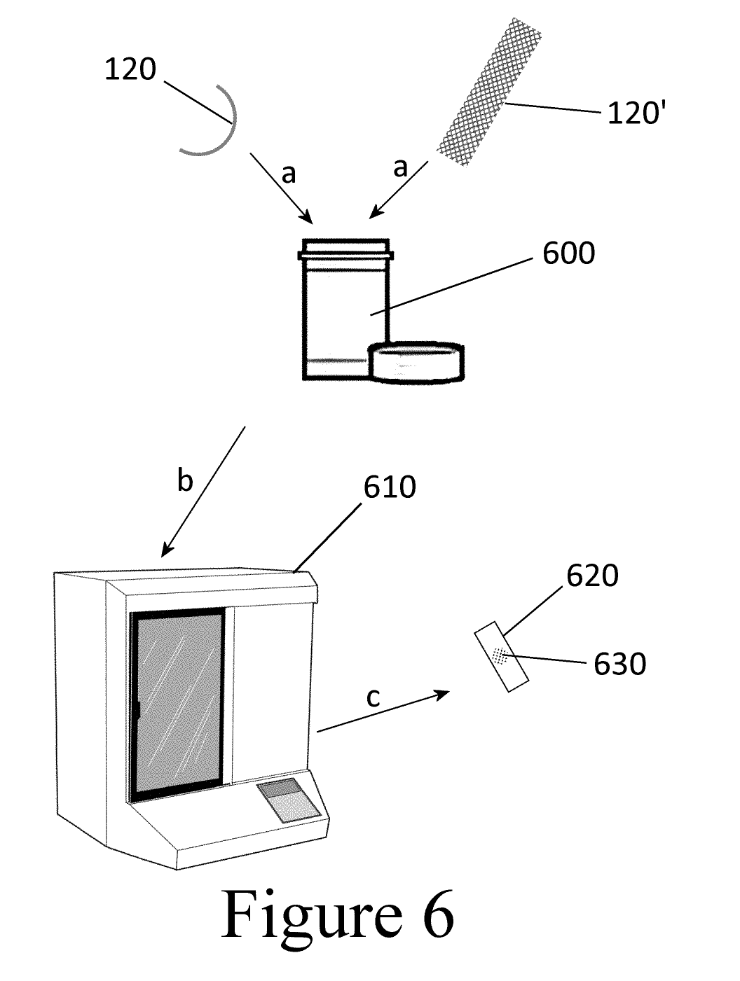

Referring to FIG. 6, there is shown a liquid-prep embodiment of the method of the invention, which may be used as an alternative to a traditional Pap smear method of simply transferring captured endometrial cells directly to a slide and staining them.

Here, an endometrial cell filter 120, or 120′ removed from a collection platform 110 is (a) placed in a preservative container 600 which, as the name implies, contains a preservative agent, such as PreservCyt®. The preservative container 600 contents may be mixed or shaken so as to substantially break up cell aggregates into individual cells.

The content of the preservative container 600 is then (b) introduced to a thin-film processor 610, such as a ThinPrep 2000®, which breaks up blood, mucus, and any non-diagnostic debris. The sample is then filtered and the cells collected onto a membrane, which membrane is then (c) fixed to a microscope slide 620 as a film layer 630 only one cell thick. The slides may then be examined microscopically for endometrial cells presenting with atypical hyperplasia or adenocarcinoma.

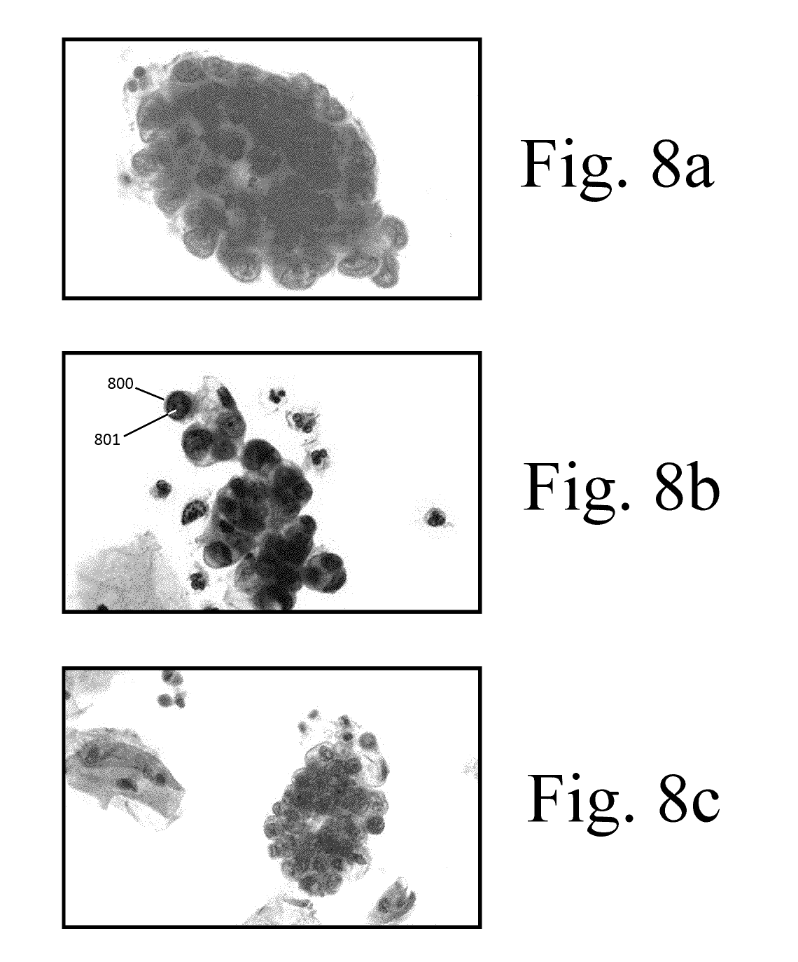

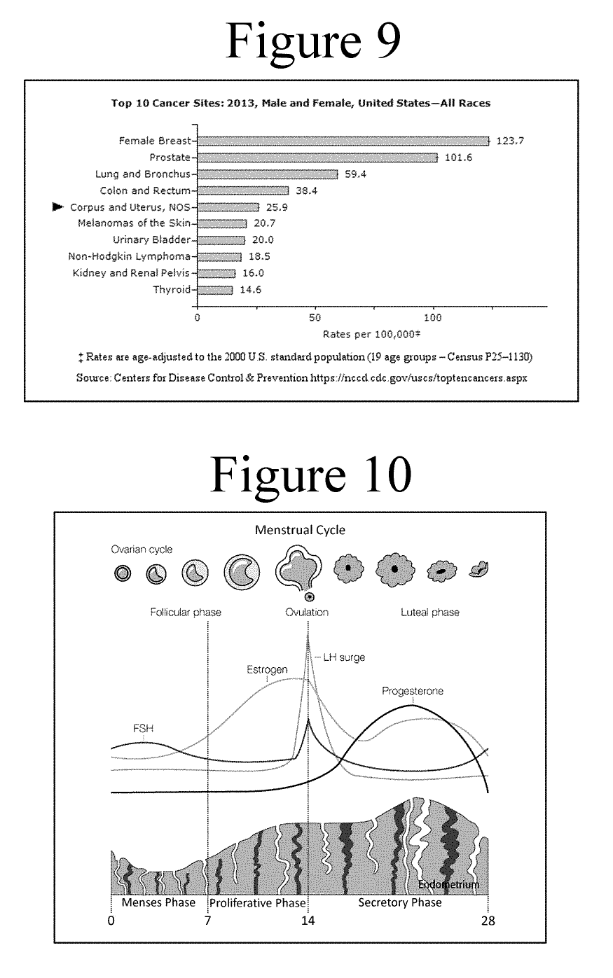

Referring to FIGS. 8, there are shown thin-layer slides of normal (benign) endometrial cells (FIG. 8a), atypical endometrial cells (FIG. 8b), and adenocarcinomic endometrial cells (FIG. 8c). Note how atypical and cancerous cells present with nuclei 801 that occupy more than half, if not nearly all, of the cell cytoplasm 800. The immediately obvious characteristic on cancerous cells is cytoplasmic eosinophilia, which refers to the strong tendency of the cell's cytoplasm to take up the bright pink dye eosin Y, as can be plainly seen in FIG. 8c.

The different colors of cytoplasm and nuclei are obtained through the use of Papanicolaou stain, which comprises (1) haematoxylin for staining nuclei, (2) Orange G for staining keratin, (3) Eosin Y, (4) Light Green SF, and optionally (5) Bismarck Brown Y, which usually stains nothing and so is often omitted.

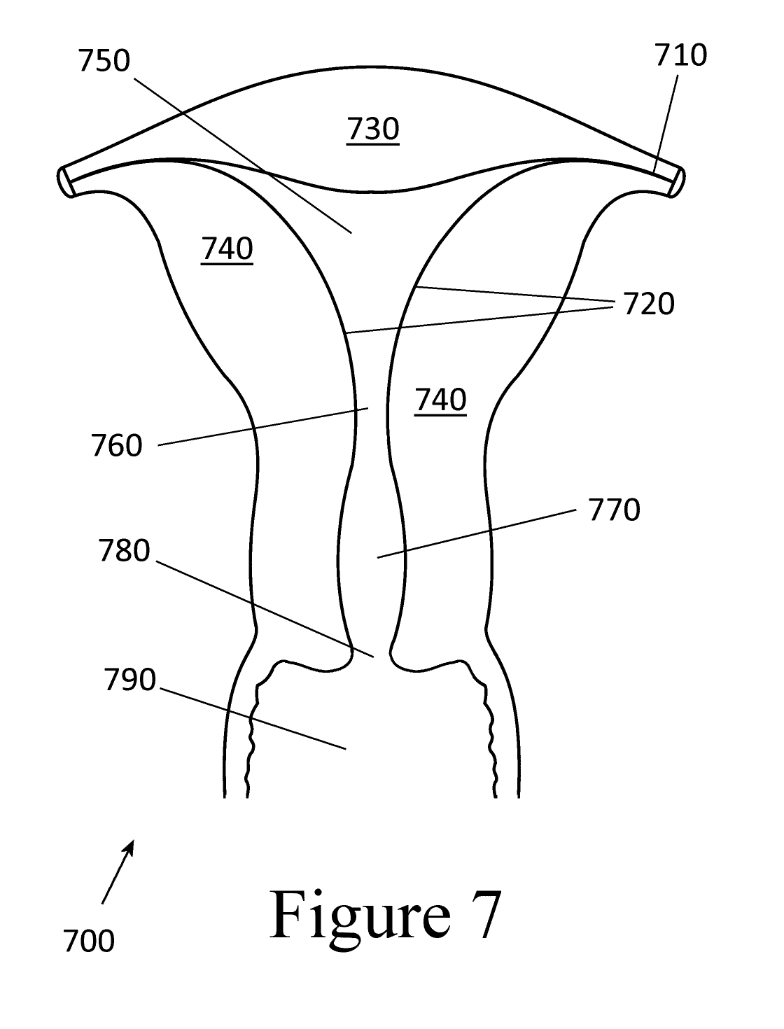

Referring back now to FIG. 7, there is shown a line drawing diagram of the human uterus 700 in cross-section for the reader's reference. Shown are the endometrium 720, the fundus 730, the uterine tubes 710, the uterine chamber 750, the myometrium (uterine wall) 740, the cervix 780, the cervical cavity 770, the internal orifice 760, and the vaginal canal 790.

The foregoing disclosures relate to illustrative embodiments of the invention and modifications may be made without departing from the spirit and scope of the invention as set forth in, and limited only by, the claims herein.

In the claims herein—unless explicitly indicated otherwise—the use of the word “or” is to be construed as the inclusive “or” in accordance with common usage in the engineering art.

What is claimed is:

1. A method of screening for endometrial disorders in a subject, comprising the steps of:
providing a menses cell capture device positioned in a menstrual flow of said subject;
recovering and identifying endometrial cells among said cell capture device; and
determining whether any of said endometrial cells present with an endometrial disorder.

2. The method of claim 1 wherein the endometrial disorder is atypical hyperplasia.

3. The method of claim 1 wherein the endometrial disorder is adenocarcinoma.

4. A menses cell capture device, comprising:
At least one endometrial cell filter having a filter substrate defining a plurality of pores sized and arrayed so as to be effective in capturing endometrial cells; and
a collection platform supporting said endometrial cell filters at least partially in the path of a menstrual flow in a configuration effective in collecting endometrial cells discharged in the menstrual flow.

5. The apparatus of claim 4 wherein the collection platform is a sanitary napkin.

6. The apparatus of claim 4 wherein said endometrial cell filter is supported in a cell basket.

7. The apparatus of claim 5 wherein the sanitary napkin further comprises a porous topsheet and said endometrial cell filter is disposed downstream therefrom.

8. The apparatus of claim 7 wherein said endometrial cell filter is in direct contact with said porous topsheet.

9. The apparatus of claim 7 further comprising a transfer layer disposed between said porous topsheet and said endometrial cell filter.

10. The apparatus of claim 4 wherein the collection platform is a tampon comprising a shaft and absorbent shaft.

11. The apparatus of claim 10 further comprising an acquisition cap disposed upstream of the endometrial cell filter.

12. The apparatus of claim 11 wherein the acquisition cap has a porosity substantially greater than that of the absorbent shaft.

13. A method of screening for endometrial disorders in a subject, comprising the steps of:
providing an endometrial cell filter at least partially positioned in a menstrual flow of said subject;
recovering and identifying endometrial cells collected in the endometrial cell filter; and
determining whether any of the collected endometrial cells present with an endometrial disorder.

14. The method of claim 13 wherein the endometrial disorder is atypical hyperplasia.

15. The method of claim 13 wherein the endometrial disorder is adenocarcinoma.

XX (canceled)

XX+1. (canceled) napkin.

XX+2. (canceled)
